SEQUENCE LISTING (19 sequences)

SEQ ID NO: 1 - DNA, 21 bp, Artificial
  primer
gctgacagaatcgttccccat

SEQ ID NO: 2 - DNA, 20 bp, Artificial
  primer
ctgaaccgtagaccaccagg

SEQ ID NO: 3 - DNA, 20 bp, artificial
  primer
acaggaagagcatgggattg

SEQ ID NO: 4 - DNA, 20 bp, Artificial
  primer
ctggtggtgtggttcatcat

SEQ ID NO: 5 - DNA, 20 bp, Artificial
  primer
taggaagaggtctccgctga

SEQ ID NO: 6 - DNA, 20 bp, Artificial
  primer
caaatgaaaaaccccaccac

SEQ ID NO: 7 - DNA, 20 bp, Artificial
  primer
cctggtggtctacggttcag

SEQ ID NO: 8 - DNA, 20 bp, Artificial
  primer
gggcaggatttgtgtctgtt

SEQ ID NO: 9 - DNA, 18 bp, Artificial
  primer
atggccgacttgctttgt

SEQ ID NO: 10 - DNA, 19 bp, Artificial
  primer
ccttggtcaccgcactttc

SEQ ID NO: 11 - protein, 337 aa, Homo sapiens [sequence omitted]

SEQ ID NO: 12 - DNA, 1011 bp, Homo sapiens
atggggaacgattctgtcagctacgagtatggggattacagcgacctctcggaccgccctgtggactgcctggatggcgcctgcctggccatcgacccgctgcgcgtggccccgctcccactgtatgccgccatcttcctggtgggggtgccgggcaatgccatggtggcctgggtggctgggaaggtggcccgccggagggtgggtgccacctggttgctccacctggccgtggcggatttgctgtgctgtttgtctctgcccatcctggcagtgcccattgcccgtggaggccactggccgtatggtgcagtgggctgtcgggcgctgccctccatcatcctgctgaccatgtatgccagcgtcctgctcctggcagctctcagtgccgacctctgcttcctggctctcgggcctgcctggtggtctacggttcagcgggcgtgcggggtgcaggtggcctgtggggcagcctggacactggccttgctgctcaccgtgccctccgccatctaccgccggctgcaccaggagcacttcccagcccggctgcagtgtgtggtggactacggcggctcctccagcaccgagaatgcggtgactgccatccggtttctttttggcttcctggggcccctggtggccgtggccagctgccacagtgccctcctgtgctgggcagcccgacgctgccggccgctgggcacagccattgtggtggggttttttgtctgctgggcaccctaccacctgctggggctggtgctcactgtggcggccccgaactccgcactcctggccagggccctgcgggctgaacccctcatcgtgggccttgccctcgctcacagctgcctcaatcccatgctcttcctgtattttgggagggctcaactccgccggtcactgccagctgcctgtcactgggccctgagggagtcccagggccaggacgaaagtgtggacagcaagaaatccaccagccatgacctggtctcggagatggaggtg

SEQ ID NO: 13 - protein, 343 aa, Mouse
MNHTTSEYYDYEYDHEHYSDLPDVPVDCPAGTCFTSDVYLIVLLVLYAAVFLVGVPGNTLVAWVTWKESRHRLGASWFLHLTMADLLCCVSLPFLAVPIAQKGHWPYGAAGCWLLSSITILSMYASVLLLTGLSGDLFLLAFRPSWKGADHRTFGVRVVQASSWMLGLLLTVPSAVYRRLLQEHYPPRLVCGIDYGGSVSAEVAITTVRFLFGFLGPLVFMAGCHGILQRQMARRHWPLGTAVVVGFFICWTPYHVLRVIIAAAPPHSLLLARVLEAEPLFNGLALAHSALNPIMFLYFGRKQLCKSLQAACHWALRDPQDEESAVTKVSISTSHEMVSEMPV

SEQ ID NO: 14 - DNA, 1029 bp, Mouse
atgaaccacaccaccagcgagtattatgactatgagtatgaccatgaacactacagtgaccttccggacgtgcccgtagactgcccagctggcacctgcttcactagtgatgtctacctgatagtcctgcttgtgctgtatgcagccgtcttcctggtgggtgtgccaggcaacaccctggtggcgtgggtgacctggaaagagtcccgccacaggcttggggcctcttggttcctgcacctgaccatggccgacttgctttgttgtgtgtctctgcccttcctggctgtgcccatcgcccaaaagggccactggccatacggggcggcaggctgctggctgttgtcctccatcaccattctgtctatgtatgccagtgtgctgctcctgaccggcctcagcggagacctcttcctactggctttcaggccctcctggaagggtgcagatcatcgaacatttggggtgcgtgtggtccaggcctcctcctggatgctgggcctcttgctgactgtgccttctgctgtctaccgtaggctgcttcaggagcactatcctccccgacttgtgtgtggcatagactatgggggatcggtcagcgccgaggttgccatcaccactgttcgattcctctttggcttcctggggccattggtgttcatggccggttgtcacggcatcctccaacggcagatggcccgccgccattggcccctgggcacagctgtcgtggtggggtttttcatttgctggaccccttatcacgtcctgcgggtaatcattgcagcggctcctccgcactccttgctcctcgcccgggtcctggaggcggagcctctgtttaatggcctggccctggctcacagtgctctcaatcccataatgtttttgtatttcggaagaaaacaactctgcaagtcactccaggctgcatgccactgggccctgagggatccacaggatgaagaaagtgcggtgaccaaggtatccatatccaccagccacgaaatggtgtctgagatgccggtg

SEQ ID NO: 15 - protein, 76 aa, homo sapiens
SVQLTEKRMDKVGKYPKELRKCCEDGMRENPMRFSCQRRTRFISLGEACKKVFLDCCNYITELRRQHARASHLGLA

SEQ ID NO: 16 - DNA, 226 bp, homo sapiens
tccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctgg

SEQ ID NO: 17 - protein, 77 aa, mouse
SVQLMERRMDKAGQYTDKGLRKCCEDGMRDIPMRYSCQRRARLITQGENCIKAFIDCCNHITKLREQHRRDHVLGLA

SEQ ID NO: 18 - DNA, 229 bp, mouse
tcagtacagttgatggaaagaaggatggacaaagctggtcagtacactgacaagggtcttcggaagtgttgtgaggatggtatgcgggatatccctatgagatacagctgccagcgccgggcacgcctcatcacccagggcgagaactgcataaaggccttcatagactgctgcaaccacatcaccaagctgcgtgaacaacacagaagagaccacgtgctgggcctgg

SEQ ID NO: 19 - DNA, 21 bp, Homo sapiens
aagaaatccaccagccatgac

(19) United States
(12) Patent Application Publication
(10) Pub. No.: US 2006/0121462 A1
(43) Pub. Date: Jun. 8, 2006

(54) Receptor and screening methods

(76) Inventors: Peter Monk, Sheffield (GB); Katherine Cianflone, L'Arcadie (CA); Allan Sniderman, Montreal (CA); David Kalant, Pointe Claire (CA); Magdalena Maslowska, La Salle (CA)

Correspondence Address: KLARQUIST SPARKMAN, LLP, 121 SW SALMON STREET, SUITE 1600, PORTLAND, OR, 97204, US

(21) Appl. No.: 10/515,799

(22) PCT Filed: May 27, 2003
(86) PCT No.: PCT/GB03/02278

§ 371 Date: Dec. 22, 2005

(51) Int. Cl. C12Q 1/68; G01N 33/53; C07H 21/02

(52) U.S. Cl. 435/6; 435/7.1; 536/23.1

(57) ABSTRACT

The invention relates to a screening method for the identification of agents which modulate the activity of a receptor, C5L2, or homologue thereof.

The invention relates to a screening method for the identification of agents which modulate the activity of a receptor, C5L2, or homologue thereof.

Obesity affects around 58 million people in the USA and contributes to the deaths of approximately 300,000 people annually (New England Journal of Medicine). Obesity adds to the risk of heart attack, stroke, diabetes and some cancers. Consequently, obesity is a severe burden on healthcare in many countries. The control of fat deposition is thought to be influenced by the hormone leptin which is encoded by the Ob gene. The level of leptin in the circulation correlates with the level of fat deposition in fat cells (adipose tissue). High levels of leptin results in greater fat deposition and a tendency to become obese. However, not all obese people have elevated levels of leptin and therefore it is thought that there may be important differences in the cause of obesity. It is thought the principal site of action of leptin is the brain, or more particularly, the hypothalamus, which controls, amongst other things, appetite.

Infection or injury leads to activation of the complement cascade, a part of the innate immune system involved in the inflammation response. For many years it has been known that fragments of the complement proteins C5, C4 and C3 are potent regulators of white blood cell activity but more recently, many non-immune cell types have been shown to respond. Only two cell surface receptors for these six fragments (C5a, C4a, C3a and their corresponding metabolites, C5adR⁷⁴, C4adR⁷⁷ and C3adR⁷⁷) have been well characterised; CD88, which binds C5a and C5adR⁷⁴ and C3aR, which binds C3a.

The 74 and 77 amino acid complement fragments, C5a and C3a, have wide ranging effects in humans. Although initially described as leukocyte chemoattractants and anaphylatoxins, it is now clear that C5a and C3a are involved in microbial host defense, immune regulation (1) and protection against toxic insult (2-5).

C5a and C3a are also reported to have psychopharmacological effects (6,7) on feeding and drinking behavior.

Both complement fragments are rapidly desarginated by serum carboxypeptidase, which modulates function. Although C5a des-Arg⁷⁴ retains most of the activity of intact C5a, albeit with a generally lower affinity for the C5a receptor (¹CD88), C3a des-Arg⁷⁷ activity is profoundly reduced relative to C3a. No binding of the des-Arg⁷⁷ form to the C3a receptor (C3aR) is observed in transfected RBL cells or mouse macrophage/monocytes (8) and, unlike C3a, C3a des-Arg⁷⁷ does not stimulate eosinophil chemotaxis (9), prostanoid production by guinea pig peritoneal macrophages and rat Kupffer cells (10) or human monocyte-like U937 cell degranulation (11). However, responses to C3a des-Arg⁷⁷ have been reported: cytotoxicity of NK cells is inhibited by both C3a and C3a des-Arg⁷⁷ (12); cytokine production by human monocyte/macrophages and PBMC is enhanced by these ligands but inhibited in human tonsil-derived B cells (13,14) and histamine release from rat peritoneal mast cells is stimulated (15).

In addition, triacylglycerol synthesis in adipocytes and preadipocytes is regulated by acylation stimulating protein (ASP), an activity shared by both C3a and C3a des-Arg⁷⁷ (16). One explanation of this pattern of responses is that cells may express two kinds of receptor: one, probably C3aR, that binds only C3a and another, as yet unidentified, that binds both C3a and C3a des-Arg⁷⁷.

A novel cbemoattractant binding protein, C5L2, that has a high affinity for C5a and C5a des-Arg⁷⁴ and a moderate affinity for C3a has recently been disclosed (17). We describe a further interaction that C5L2 also binds C3a des-Arg⁷⁷/ASP and is expressed in human adipose tissue.

The expression of C5L2 in adipose tissue and its binding to C3a des-Arg⁷⁷/ASP describes a novel interaction which has utility with respect to the identification of agents which either mimic C3a des-Arg⁷⁷/ASP activation of C5L2 or inhibit the interaction with a view to providing antagonists of C5L2 activation. Agents obtainable by the method have utility with respect to modulating fat deposition in adipocytes.

According to an aspect of the invention there is provided a screening method for the identification of agents which modulate the activity of the receptor C5L2, or homologue thereof.

According to an aspect of the invention there is provided a screening method for the identification of agents which modulate receptor activity comprising the steps of:
i) forming a preparation comprising a polypeptide, or active fragment thereof, encoded by a nucleic acid molecule selected from the group consisting of:
   a) a polypeptide encoded by a nucleic acid molecule as represented by the nucleic acid sequence in FIG. 7;
   b) a polypeptide encoded by a nucleic acid molecule which hybridizes to the nucleic acid sequence in FIG. 7 and which has the activity associated with the receptor C5L2;
   c) a polypeptide encoded by a nucleic acid molecule which has a nucleic acid sequence which is degenerate because of the genetic code to the sequences in (a) and (b); and a candidate agent to be tested; and
ii) detecting or measuring the effect of the agent on the activity of said polypeptide.

In a preferred method of the invention said preparation includes a second polypeptide encoded by a nucleic acid molecule selected from the group consisting of:

i) a polypeptide encoded by a nucleic acid molecule as represented in FIG. 11;
ii) a polypeptide encoded by a nucleic acid molecule which hybridizes to the sequence in FIG. 11 and which has the activity of C3a des-Arg⁷⁷/ASP; and
iii) a polypeptide encoded by a nucleic acid molecule which has a nucleic acid sequence which is degenerate because of the genetic code to the sequences in (i) and (ii).

In a preferred method of the invention there is provided a nucleic acid molecule which anneals under stringent hybridisation conditions to the sequences described in (a) and (i) above.

Stringent hybridisation/washing conditions are well known in the art. For example, nucleic acid hybrids that are stable after washing in 0.1×SSC, 0.1% SDS at 60° C. It is well known in the art that optimal hybridisation conditions can be calculated if the sequence of the nucleic acid is known. Typically, hybridisation conditions uses 4-6×SSPE (20×SSPE contains 175.3 g NaCl, 88.2 g NaH₂PO₄H₂O and 7.4 g EDTA dissolved to 1 litre and the pH adjusted to 7.4); 5-10× Denhardts solution (50× Denhardts solution contains 5 g Ficoll (Type 400, Pharmacia), 5 g polyvinylpyrrolidone abd 5 g bovine serum albumen; 100 μg-1.0 mg/ml sonicated salmon/herring DNA; 0.1-1.0% sodium dodecyl sulphate; optionally 40-60% deionised formamide. Hybridisation temperature will vary depending on the GC content of the nucleic acid target sequence but will typically be between 42°-65° C.

In a preferred method of the invention said polypeptide, or active binding fragment thereof, is encoded by a nucleic acid molecule comprising the sequence in FIG. 7 or 11. In a further preferred method of the invention said nucleic acid molecule consists of the sequence in FIG. 7 or 11.

In a preferred method of the invention said polypeptide is modified by deletion, subsitution or addition of at least one amino acid residue of the sequence represented in FIG. 6.

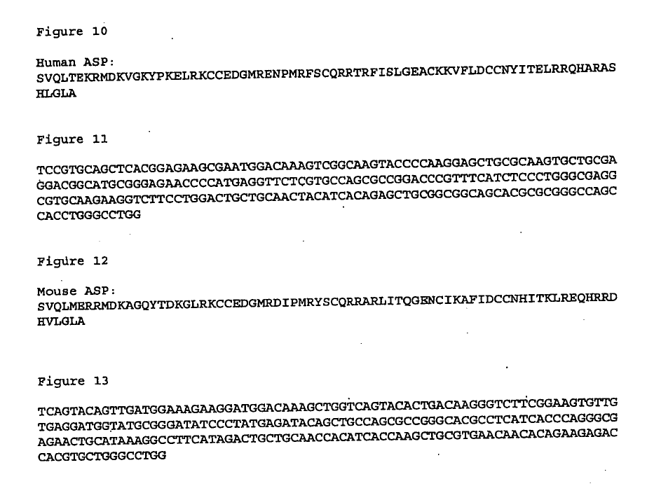

In a further preferred method of the invention said second polypeptide is modified by deletion, substitution or addition of at least one amino acid residue of the sequence represented in FIG. 10.

A modified or variant, i.e. a fragment polypeptide and reference polypeptide, may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan. Preferred are variants which retain the same biological function and activity as the reference polypeptide from which it varies Alternatively, variants include those with an altered biological function, for example variants which act as antagonists.

Alternatively or in addition, non-conservative substitutions may give the desired biological activity see Cain S A, Williams D M, Harris V, Monk P N. Selection of novel ligands from a whole-molecule randomly mutated C5a library. Protein Eng. 2001 March; 14(3):189-93, which is incorporated by reference.

A functionally equivalent polypeptide according to the invention is a variant wherein one in which one or more amino acid residues are substituted with conserved or non-conserved amino acid residues, or one in which one or more amino acid residues includes a substituent group. Conservative substitutions are the replacements, one for another, among the aliphatic amino acids Ala, Val, Leu and Ile; interchange of the hydroxl residues Ser and Thr; exchange of the acidic residues Asp and Glu; substitution between amide residues Asn and Gln; exchange of the basic residues Lys and Arg; and replacements among aromatic residues Phe and Tyr.

In addition, the invention features polypeptide sequences having at least 75% identity with the polypeptide sequences as hereindisclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated herein.

In a further preferred method of the invention said polypeptide and second polypeptide is/are expressed by a cell, preferably a mammalian cell, for example and not by limitation, a CHO cell, a COS cell. Preferably said cell is an adipocyte Preferably said cell is genetically engineered to express said polypeptide.

In a preferred method of the invention said agent is selected from the group consisting of: a polypeptide; a peptide; an aptamer.

In a preferred method of the invention said polypeptide is an antibody.

In a preferred method of the invention said antibody is a polyclonal or monoclonal antibody.

Antibodies, also known as immunoglobulins, are protein molecules which have specificity for foreign molecules (antigens). Immunoglobulins (Ig) are a class of structurally related proteins consisting of two pairs of polypeptide chains, one pair of light (L) (low molecular weight) chain (K or λ), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant.

The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region.

The H chains of Ig molecules are of several classes, α, μ, σ, α, and γ (of which there are several sub-classes). An assembled Ig molecule consisting of one or more units of two identical H and L chains, derives its name from the H chain that it possesses. Thus, there are five Ig isotypes: IgA, IgM, IgD, IgE and IgG (with four sub-classes based on the differences in the H chains, i.e., IgG1, IgG2, IgG3 and IgG4). Further detail regarding antibody structure and their various functions can be found in, Using Antibodies: A laboratory manual, Cold Spring Harbour Laboratory Press.

Chimeric antibodies are recombinant antibodies in which all of the V-regions of a mouse or rat antibody are combined with human antibody C-regions. Humanised antibodies are recombinant hybrid antibodies which fuse the complimentarity determining regions from a rodent antibody V-region with the framework regions from the human antibody V-regions. The C-regions from the human antibody are also used. The complimentarity determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not illicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

It will be apparent that the antibody could be specific for C5L2 or C3a des-Arg⁷⁷/ASP. Furthermore said antibodies can be agonistic or antagonistic.

In a preferred method of the invention said peptide is an oligopeptide. Preferably, said oligopeptide is at least 10 amino acids long. Preferably said oligopeptide is at least 20, 30, 40, 50 amino acids in length.

In a further preferred method of the invention said peptide is a modified peptide.

Peptides or protein fragments can be derived from the receptor, or the intact receptor expressed in a suitable cell line as a means of selecting novel oligopeptide ligands from peptide libraries see, [Faibrother W J, Christinger H W, Cochran A G, Fuh G, Keenan C J, Quan C, Shriver S K, Tom J Y, Wells J A, Cunningham B C. Novel peptides selected to bind vascular endothelial growth factor target the receptor-binding site. Biochemistry. 1998 Dec. 22; 37(51):17754-64.] or mutant complement fragment libraries displayed on phage [Cain S A, Williams D M, Harris V, Monk P N. Selection of novel ligands from a whole-molecule randomly mutated C5a library. Protein Eng. 2001 March;14(3):189-93.]

It will be apparent to one skilled in the art that modified amino acids include, by way of example and not by way of limitation, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, ornithine. Other modifications include amino acids with a C₂, C₃ or C₄ alkyl R group optionally substituted by 1, 2 or 3 substituents selected from halo (eg F, Br, I), hydroxy or C₁-C₄ alkoxy.

Alternatively said peptide is modified by acetylation and/or amidation.

In a preferred method of the invention the polypeptides or peptides are modified by cyclisation. Cyclisation is known in the art, (see Scott et al Chem Biol (2001), 8:801-815; Gellerman et al J. Peptide Res (2001), 57: 277-291; Dutta et al J. Peptide Res (2000), 8: 398-412; Ngoka and Gross J Amer Soc Mass Spec (1999), 10:360-363).

In a further preferred method of the invention said agent is an aptamer.

Nucleic acids and proteins have both linear sequence structure, as defined by their base or amino acid sequence, and also a three dimensional structure which in part is determined by the linear sequence and also the environment in which these molecules are located. Conventional therapeutic molecules are small molecules, for example, peptides, polypeptides, or antibodies, which bind target molecules to produce an agonistic or antagonistic effect. It has become apparent that nucleic acid molecules also have potential with respect to providing agents with the requisite binding properties which may have therapeutic utility. These nucleic acid molecules are typically referred to as aptamers. Aptamers are small, usually stabilised, nucleic acid molecules which comprise a binding domain for a target molecule.

In a further preferred method of the invention said aptamer comprises at least one modified nucleotide base.

The term “modified nucleotide base” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include by example and not by way of limitation; alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N-6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N-6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil;]-methylguanine; 1-methylcytosine;

The aptamers of the invention are synthesized using conventional phosphodiester linked nucleotides and synthesized using standard solid or solution phase synthesis techniques which are known in the art. Linkages between nucleotides may use alternative linking molecules. For example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

In a further preferred method of the invention said agent is an inhibitory RNA (RNAi) molecule.

A technique to specifically ablate gene function is through the introduction of double stranded RNA, also referred to as inhibitory RNA (RNAi), into a cell which results in the destruction of mRNA complementary to the sequence included in the RNAi molecule. The RNAi molecule comprises two complementary strands of RNA (a sense strand and an antisense strand) annealed to each other to form a double stranded RNA molecule. The RNAi molecule is typically derived from exonic or coding sequence of the gene which is to be ablated.

Preferably said RNAi molecule is derived from the nucleic acid molecule comprising a nucleic acid sequence selected from the group consisting of;
a) a nucleic acid sequence as represented by the sequence in FIG. 7, or fragment thereof;
b) a nucleic acid sequence which hybridises to the nucleic acid sequences of FIG. 7 and encodes a gene for the C5L2 receptor;
c) a nucleic acid sequence which comprise sequences which are degenerate as a result of the genetic code to the nucleic acid sequences defined in (a) and (b).

Recent studies suggest that RNAi molecules ranging from 100-1000 bp derived from coding sequence are effective inhibitors of gene expression. Surprisingly, only a few molecules of RNAi are required to block gene expression which implies the mechanism is catalytic. The site of action appears to be nuclear as little if any RNAi is detectable in the cytoplasm of cells indicating that RNAi exerts its effect during mRNA synthesis or processing.

The exact mechanism of RNAi action is unknown although there are theories to explain this phenomenon. For example, all organisms have evolved protective mechanisms to limit the effects of exogenous gene expression. For example, a virus often causes deleterious effects on the organism it infects. Viral gene expression and/or replication therefore needs to be repressed. In addition, the rapid development of genetic transformation and the provision of transgenic plants and animals has led to the realisation that transgenes are also recognised as foreign nucleic acid and subjected to phenomena variously called quelling (Singer and Selker, 1995), gene silencing (Matzke and Matzke, 1998), and co-suppression (Stam et. al., 2000).

Initial studies using RNAi used the nematode Caenorhabditis elegans. RNAi injected into the worm resulted in the disappearance of polypeptides corresponding to the gene sequences comprising the RNAi molecule (Montgomery et al., 1998; Fire et al., 1998). More recently the phenomenon of RNAi inhibition has been shown in a number of eukaryotes including, by example and not by way of limitation, plants, trypanosomes (Shi et al., 2000) Drosophila spp. (Kennerdell and Carthew, 2000). Recent experiments have shown that RNAi may also function in higher eukaryotes. For example, it has been shown that RNAi can ablate c-mos in a mouse ooctye and also E-cadherin in a mouse preimplanation embryo (Wianny and Zemicka-Goetz, 2000).

More preferably said RNAi molecule according has a length of between 10 nucleotide bases (nb)-1000 nb. Even more preferably said RNAi molecule has a length of 10 nb; 20 nb; 30 nb; 40 nb; 50 nb; 60 nb; 70 nb; 80 nb; 90 nb; or 100 bp. Even more preferably still said RNAi molecule is 21 nb in length.

Even more preferably still the RNAi molecule comprises the nucleic acid sequence AAGAAATCCACCAGCCATGAC.

Even more preferably still the RNAi molecule consists of the nucleic acid sequence AAGAAATCCACCAGCCATGAC

The RNAi molecule may comprise modified nucleotide bases.

In a still further preferred method of the present invention an antisense oligonucleotide is provided which is capable of hybridising to the nucleic acid molecule encoding the C5L2 receptor.

Even more preferably this antisense oligonucleotide is capable of hybridising to the nucleic acid molecule encoding the human C5L2 receptor. Preferably said antisense oligonucleotide comprises a nucleic acid sequence selected from the group consisting of;

GCTGACAGAATCGTTCCCCAT;
CTGAACCGTAGACCACCAGG;
ACAGGAAGAGCATGGGATTG.

Alternatively, said antisense oligonucleotide consists a nucleic acid sequence selected from the group consisting of;

GCTGACAGAATCGTTCCCCAT;
CTGAACCGTAGACCACCAGG;
ACAGGAAGAGCATGGGATTG.

In a further preferred embodiment of the present invention, said antisense oligonucleotide is capable of hybridising to the nucleic acid molecule encoding the mouse C5L2 receptor. Preferably said antisense oligonucleotide comprises a nucleic acid sequence selected from the group consisting of;

CTGGTGGTGTGGTTCATCAT;
TAGGAAGAGGTCTCCGCTGA;
CAAATGAAAAACCCCACCAC.

Alternatively, said antisense oligonucleotide consists a nucleic acid sequence selected from the group consisting of;

CTGGTGGTGTGGTTCATCAT;
TAGGAAGAGGTCTCCGCTGA;
CAAATGAAAAACCCCACCAC.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and thereby, inhibits the transcription of that gene and/or the translation of that mRNA. The antisense molecules are designed so as to interfere with transcription or translation of a target gene upon hybridization with the target gene. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions.

In order to be sufficiently selective and potent for inhibition, such antisense oligonucleotides should comprise at least 7 (Wagner et al., Nature Biotechnology 14:840-844, 1996) and more preferably, at least 15 consecutive bases which are complementary to the target. Most preferably, the antisense oligonucleotides comprise a complementary sequence of 20-30 bases.

Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted. The 3′-untranslated regions are known to contain cis acting sequences which act as binding sites for proteins involved in stabilising mRNA molecules. These cis acting sites often form hair-loop structures which function to bind said stabilising proteins. A well known example of this form of stability regulation is shown by histone mRNA's, the abundance of which is controlled, at least partially, post-transcriptionally.

The term “antisense oligonucleotides” is to be construed as materials manufactured either in vitro using conventional oligonucleotide synthesising methods which are well known in the art or oligonucleotides synthesised recombinantly using expression vector constructs.

The present invention, thus, contemplates pharmaceutical preparations containing natural and/or modified antisense molecules that are complementary to and hybridizable with, under physiological conditions, nucleic acids encoding proteins the regulation of results in beneficial therapeutic effects, together with pharmaceutically acceptable carriers (eg polymers, liposomes/cationic lipids).

Antisense oligonucleotides may be administered as part of a pharmaceutical composition. Such a pharmaceutical composition may include the antisense oligonucleotides in combination with any standard physiologically and/or pharmaceutically acceptable carriers which are known in the art (eg liposomes). The compositions should be sterile and contain a therapeutically effective amount of the antisense oligonucleotides in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism.

According to a further aspect of the invention there is provided an agent obtainable by the method according to the invention.

Preferably said agent is an agonist of C5L2 receptor activation. Alternatively said agent is an antagonist of C5L2 receptor activation.

In a preferred embodiment of the invention there is provided the use of said agent as a pharmaceutical.

In a further preferred embodiment of the invention there is provided the use of an agent according to the invention for the manufacture of a medicament for use in the treatment of obesity.

According to a further aspect of the invention there is provided a method to treat obesity comprising administering to an animal, preferably a human, an agent according to the invention.

According to a still further aspect of the present invention there is provided the use of a PI-3 inhibitor for the manufacture of a medicament for use in the treatment of obesity. Preferably this PI-3 inhibitor is wortmanin or a functional variant thereof.

An embodiment of the invention will now be described by example only and with reference to the following figures:

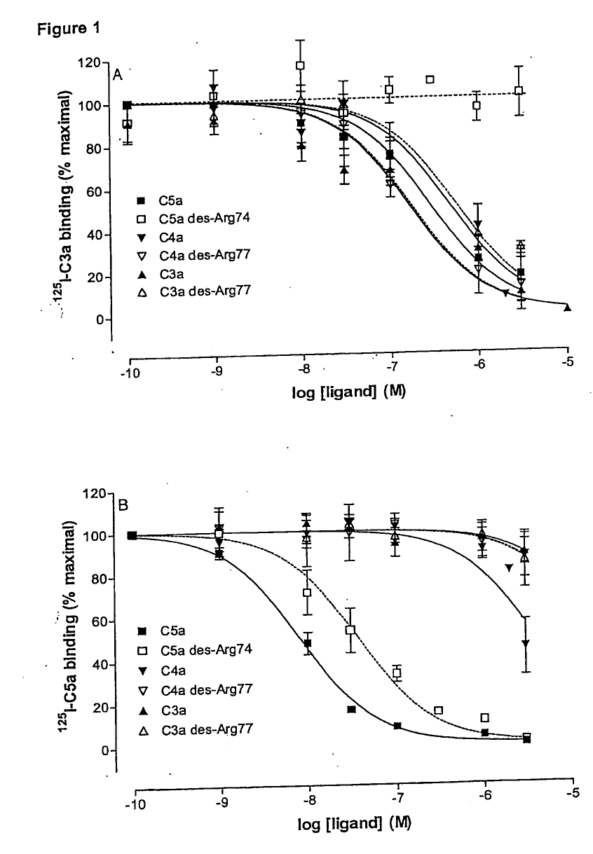

FIG. 1 illustrates C3a des-Arg⁷⁷ and C4a des-Arg⁷⁷ bind to RBL cells expressing C5L2. RBL cells transfected with C5L2 were incubated with the stated concentrations of complement fragments for 10 min prior to the addition of 50 pM ¹²⁵I-C3a (A) or 50 pM ¹²⁵I-C5a (B). Results are the means of n (n shown in Table 1) separate experiments performed in duplicate±S.E.;

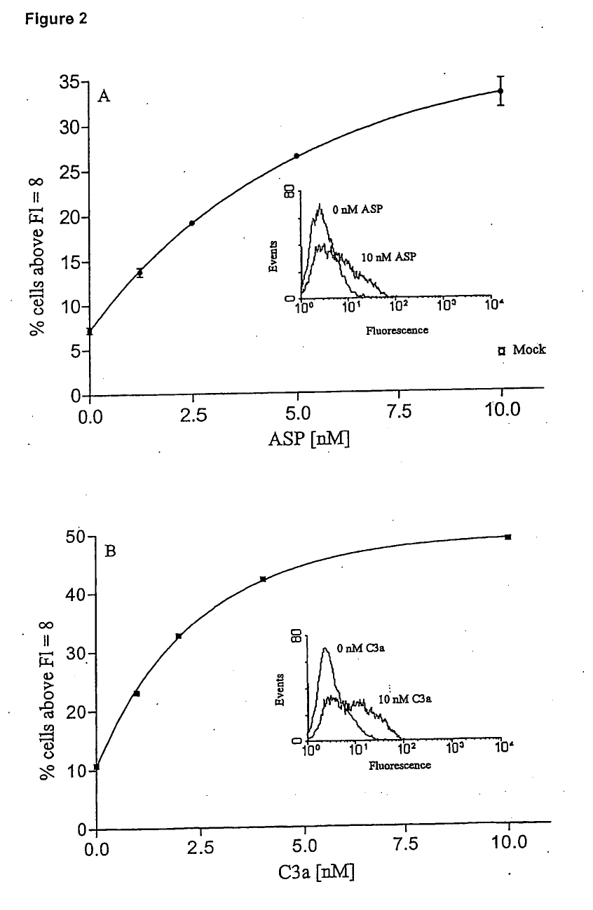

FIG. 2 illustrates C5L2 shows saturable binding/uptake of ASP. HEK 293 cells were transfected with C3aR (A) or C5L2 (B) and three days later cells were incubated for 30 minutes with the indicated concentrations of FLUOS-labelled C3a or ASP, respectively. Binding/uptake was assessed by FACS, and the percentage of cells above a fluorescence intensity of 8 was determined. The fluorescence histograms at the highest and lowest ligand concentrations are shown in the insets;

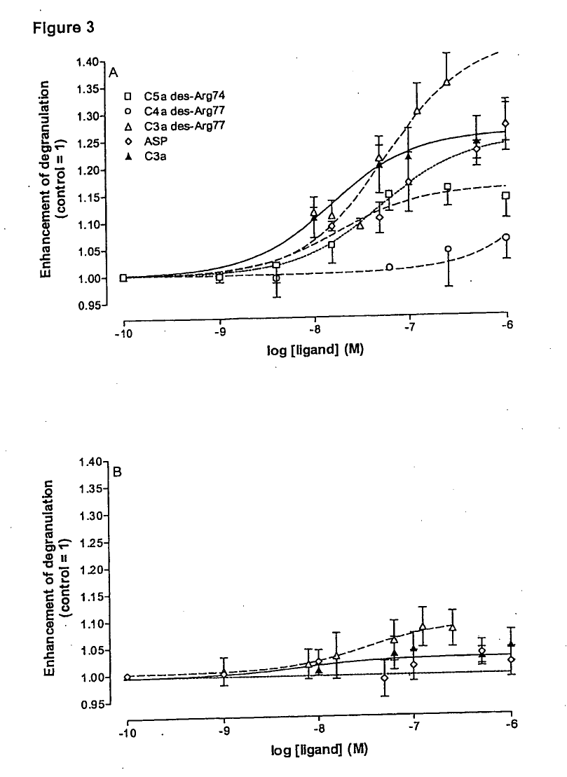

FIG. 3 illustrates C3a des-Arg⁷⁷ enhances the degranulation response to cross-linkage of the high affinity IgE receptor in cells transfected with C5L2. RBL cells transfected with A), C5L2 or B), an irrelevant receptor (human formyl peptide-like receptor-1) were incubated overnight with 1 g/ml IgE^{DNP} and then treated with the stated concentrations of C5a des-Arg⁷⁴, C4a des-Arg⁷⁷ or C3a des-Arg⁷⁷ or ASP for 15 minutes prior to the addition of the cross-linking agent, HSA-DNP at 100 ng/ml. Degranulation was assessed as the secretion of β-hexosaminidase. Results are shown as a percentage of the release stimulated by 100 ng/ml HSA-DNP in the absence of anaphylatoxin (control=1) and are means of 3-6 separate experiments performed in triplicate, ±S.E.

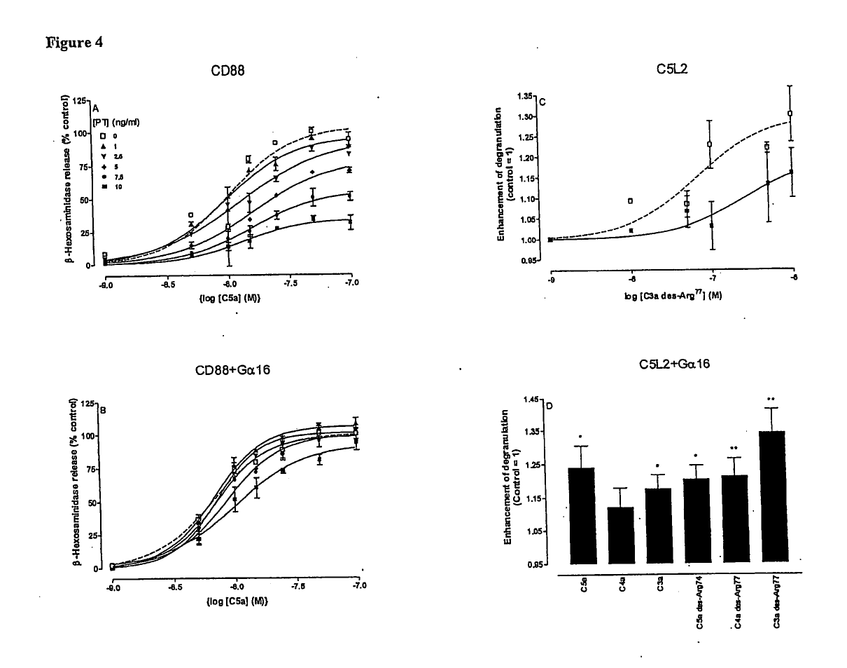

FIG. 4 illustrates CD88 and C5L2 couple to pertussis toxin-sensitive and insensitive G proteins. RBL cells were transfected with human CD88 (A), CD88+G_{α16} (B), C5L2 (C) or C5L2+G_{α16} (D) using a monocistronic or a bicistronic expression vector. Functional association of CD88 or C5L2 with G_{α16} was demonstrated using pertussis toxin treatment to inhibit endogenous Gᵢ-like G proteins: CD88 (A) and CD88+G_{α16} (B) transfected RBL cells were treated for 4 h with 0-10 ng/ml pertussis toxin (PT) prior to the addition of 50 nM C5a. RBL cells transfected with C5L2 (C) were treated with either medium alone (open squares) or 10 ng/ml PT (solid squares) for 4 h prior to the addition of purified human ASP/C3a des-Arg⁷⁷. RBL cells co-transfected with C5L2+G_{α16} (D) were incubated with 1 M of the stated complement fragment after treatment with 10 ng/ml pertussis toxin for 4 h. For C and D, treatment for 15 min with anaphylatoxins preceded the determination the enhancement of the degranulation response to IgE cross-linking. Results are the means of at least three experiments performed in triplicate±S.E. Significantly different from control (=1): *P<0.05; **P<0.01; (one-tailed t test).

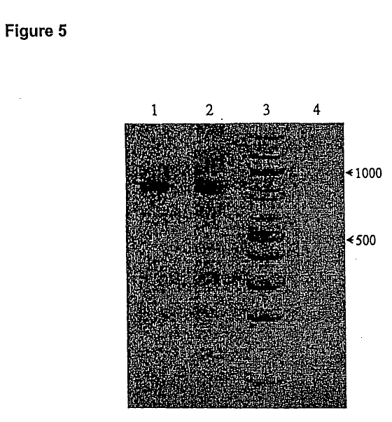

FIG. 5 illustrates C5L2 is expressed in human adipose tissue and murine preadipocytes. RT-PCR of human adipose tissue and mouse preadipocyte 3T3-L1 cells with primers to C5L2 show bands of expected size after polyacrylamide gel electrophoresis/silver staining. Lanes 1 and 2; two sources of human adipose tissue. Lane 3; 100 bp ladder with 500 and 1000 bp indicated. Lane 4; 3T3-L1 preadipocytes;

FIG. 6 is the amino acid sequence of human C5L2;

FIG. 7 is the DNA sequence of human C5L2;

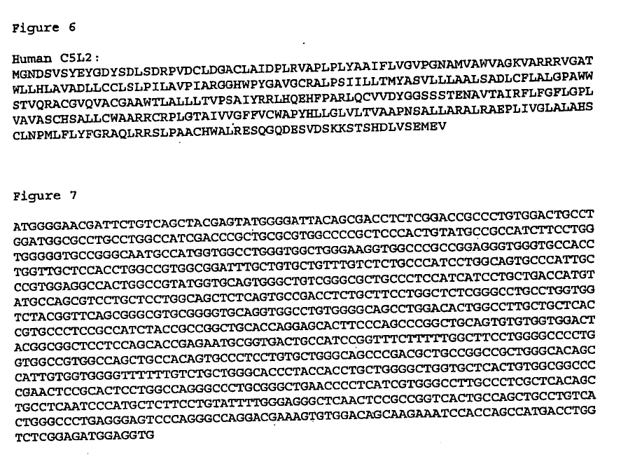

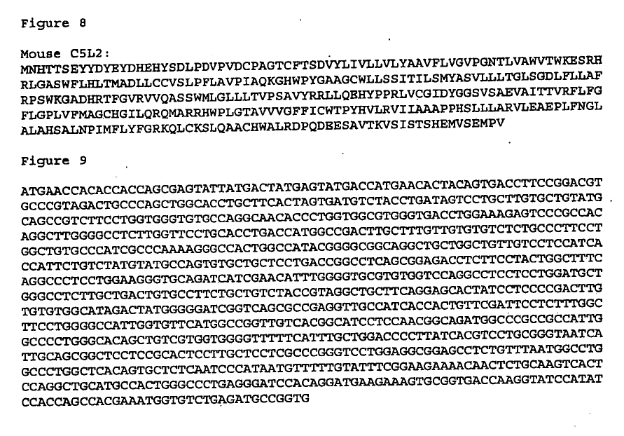

FIG. 8 is the amino acid sequence of murine C5L2;

FIG. 9 is the DNA sequence of murine C5L2;

FIG. 10 is the amino acid sequence of human ASP/C3a des-Arg⁷⁷;

FIG. 11 is the DNA sequence of human ASP/C3a des-Arg⁷⁷;

FIG. 12 is the amino acid sequence of murine ASP/C3a des-Arg⁷⁷; and

FIG. 13 is the DNA sequence of murine ASP/C3a des-Arg⁷

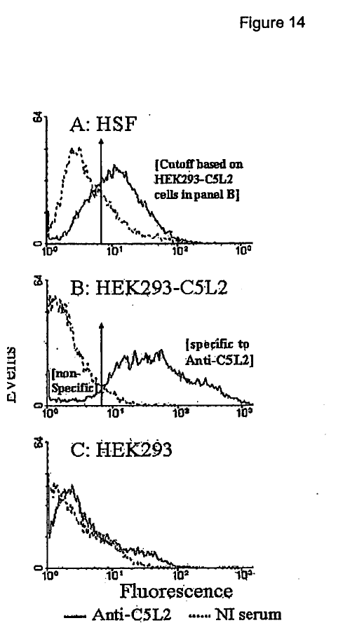

FIG. 14. Human Fibroblasts Demonstrate Cell Surface Expression of Human C5L2. A rabbit polyclonal antibody was generated against a peptide representing a portion of the N-terminal extracellular region of C5L2. HSF cells (A), HEK 293 cells stably transfected with C5L2 (B), and non-transfected HEK 293 cells (C) were detached non-enzymatically and incubated at 4° C. with either rabbit anti-C5L2 (solid line) or rabbit non-immune serum (NI; dashed line) as control. After washing, the cells were incubated with goat anti-rabbit IgG conjugated to FITC. After washing and fixing with paraformaldehyde, cellular fluorescence was measured by FACS. HSF (and 3T3, not shown) have much greater autofluoresence than HEK cells, resulting in greater background interference.

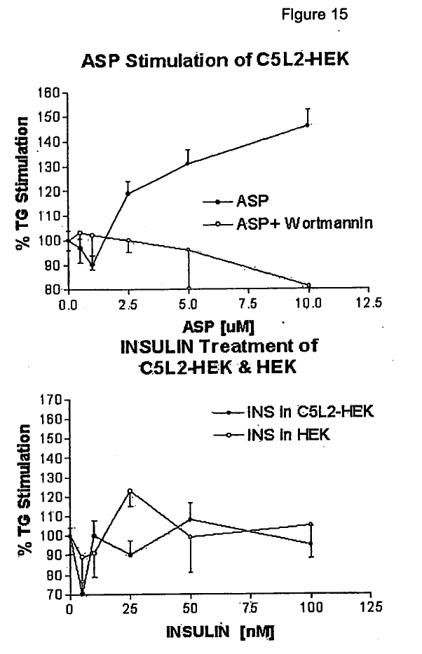

FIG. 15: Functional Response of HEK293 Cells Stably Transfected with C5L2 to ASP and Insulin: Cells were tested for response to ASP and insulin stimulation of triglyceride synthesis with 100 μM ³H oleate for 4 hours. Results are calculated as nmol ³H oleate incorporated into triglyceride/mg cell protein where 0 ASP is set as 100%. ASP stimulation in C5L2-HEK cells is comparable to that in HSF and 3T3 and comparable to the level of insulin stimulation of TGS (J Biol Chem in press). Transfection with C5L2 does not render the cells insulin responsive for TGS although it does render them ASP responsive In addition, treatment with wortmannin blocks the ASP effect as it does in 3T3 cells which normally respond to ASP, and express C5L2 (unpublished). As in many biologic assays, the concentration required for activity at 37° C. under dynamic conditions is greater than the binding affinity (which is assessed at 4° C. under static conditions). For example, insulin Kd is in the pM range, but nM concentrations are required for activity. ASP Kd is high nM range, but biologic activity is in low μM range. By contrast, non-transfected HEK cells do not respond to either ASP or insulin.

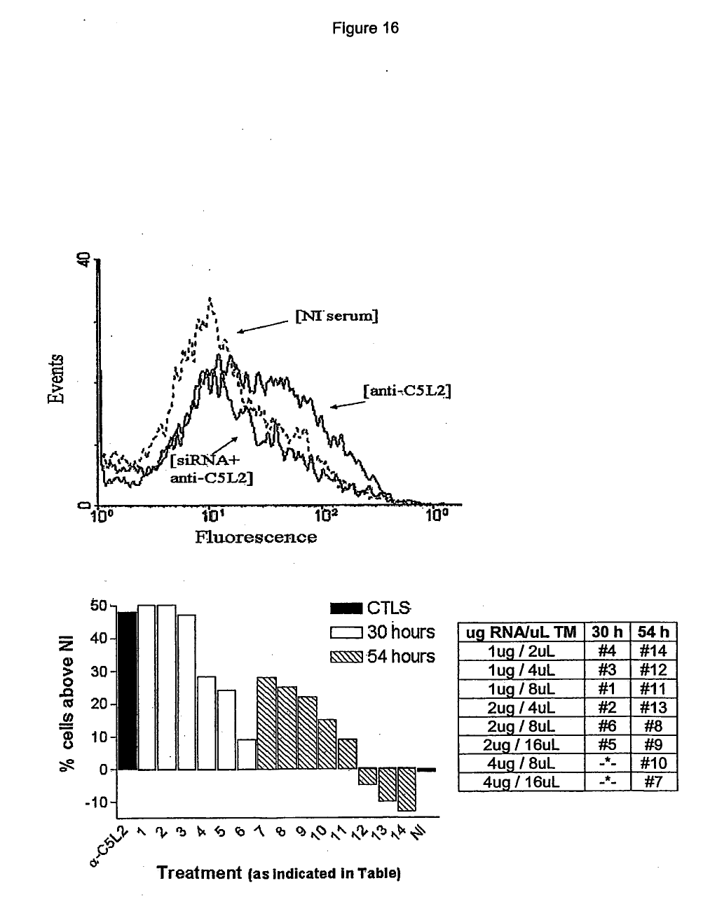

FIG. 16: C5L2 Expression in HSF is Downregulated by Interfering RNA Treatment: Interfering RNA was transfected into 80-90% confluent HSF cells using Qiagen “Transmessenger” kit according to manufacturer's instructions. Interfering RNA was designed according to manufacturer's guidelines. Non-silencing siRNA (provided by Qiagen) was used as control. Following incubation for 30 or 54 hours, cells were treated as described in FIG. 14 with anti-human C5L2 for FACS analysis. Top Panel: FACS analysis of HSF treated with non-immune serum, anti-C5L2 antibody and anti-C5L2+siRNA treatment (treatment #12 in table below). Bottom panel: the percentage of cells above the cutoff point (as shown in FIG. 14) was used to evaluate the relative C5L2 expression according to the treatment condition (see table) as compared to mock treatment (anti-C5L2) and negative control with non-immune serum (NI). Various conditions were assessed: 14 μg RNA, 2-16 μL transmessenger (TM) reagent and 30 vs 54 hours, where *=too few viable cells to analyze.

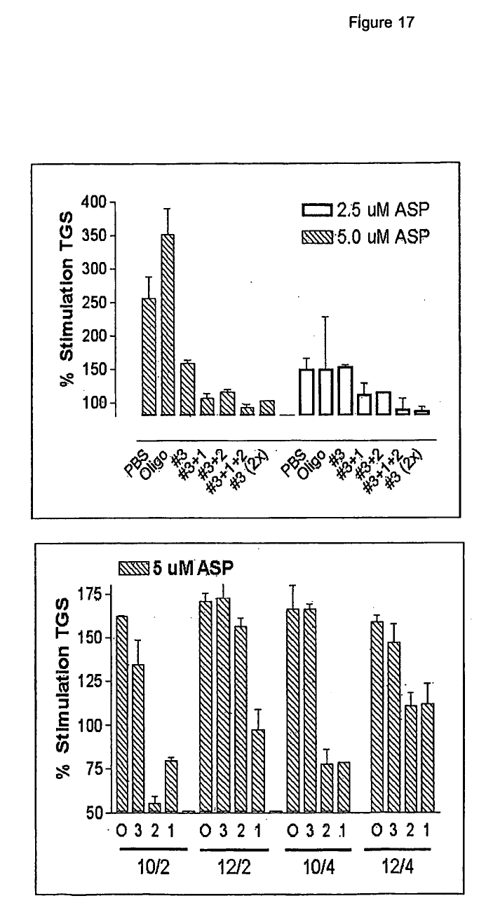

FIG. 17: Antisense Treatment Reduces Response to ASP in Human HSF and Murine 3T3 Cells: Antisense oligos were designed against three separate regions of RNA sequence. A non-sense oligo was also designed for use as a control. Oligos were transfected into 30-50% confluent HSF or 3T3 cells using “Oligofectamine” according to manufacturer's instructions. Following incubation for 48 hours, cells were analyzed for basal and ASP-stimulated triglyceride synthesis. For each oligo treatment, triglyceride synthesis in cells without ASP was set as =100%. ASP induced stimulation above 100% is shown in the graphs below. Top Panel: HSF cells treated with 2 μg RNA and 12 μL oligofectamine, then tested for TGS stimulation with 0, 2.5 and 5.0 μM ASP for 4 hours. PBS=mock-transfection, Oligo=nonsense oligo, #1,#2,#3=anti-RNA oligos for human, Basal TGS=100%. Bottom Panel: 3T3 cells treated with indicated amounts of Oligofectamine (μL)/RNA (μg) (ratios of 10/2 to 12/4) then tested for TGS with 0 or 5 μM ASP, O=nonsense oligo, #1,#2, #3=anti-RNA oligos for murine. Basal TGS=100%.

### MATERIALS AND METHODS

Cell Lines and Culture Conditions

RBL-2H3 and HEK 293 cells were routinely cultured in Dulbecco's modified Eagle's medium+10% (v/v) fetal calf serum at 37° C., 5% CO₂. The media was supplemented with 400 mg/L G-418 for stably transfected cells.

Stable Transfection of RBL Cells.

C5L2, C3aR and CD88-transfected RBL cells were produced as described (17). G₁₆ was cloned from human monocytes mRNA and authenticated by sequencing. Human G₁₆ and either C5L2 or CD88 were ligated into the bicistronic expression vector pIRES (Qiagen). Stable transfection of RBL-2H3 cells with pIRES constructs was achieved by electroporation (18). Cells underwent three rounds of fluorescence-activated cell sorting using anti-CD88 antibody (clone S5/1; Serotec) or anti-hemagglutinin peptide antibody (Roćhe Molecular Biochemicals, clone 12CA5), selecting the top 5% of receptor-positive cells in each round.

Transient Transfection of HEK 293 Cells

HEK 293 cells were seeded into 6-well plates at 1×10⁶ cells/well the day before transfection. C5L2 in vector pEE6hCMV.neo (Celltech) or C3aR in vector pcDNA1/AMP (Invitrogen) at 2 μg DNA/well were transfected with Lipofectamine2000 (5 μl/well) (Invitrogen) according to the manufacturer's protocol. Cells were assayed for binding/uptake three days post-transfection.

Production of Anaphylatoxins

Expression and purification of the recombinant His₆-tagged C5a, C5a des-Arg⁷⁴ and C3a was performed under denaturing conditions as described (19). Recombinant C4a, C4a des-Arg⁷⁷ and C3a des-Arg⁷⁷ were expressed and purified under non-denaturing conditions by sonication in the presence of BugBuster Protein Extraction Reagent (Novagen) using manufacturer's conditions. Plasma ASP (C3a des-Arg⁷⁷) and plasma C3a were purified as previously described (16).

Fluorescent Labelling of ASP and C3a

ASP or C3a was labelled with FLUOS (Roche Biochemicals) at a molar ratio of 1:10 (ligand:FLUOS) for two hours according to the manufacturer's recommendations. Labelled ligand was separated from free FLUOS on a Sephadex G25M column and stored in aliquots at −80° C.

Cellular Activation Assays

Cellular activation was measured as the release of -hexosaminidase from intracellular granules (20). In the enhancement of degranulation assay, cells were treated with 1 μg/ml IgE^{DNP} (21) overnight, then with chemoattractants or peptides for 10 min prior to stimulation with 100 ng/ml DNP-HSA (Sigma) for 15 minutes. EC₅₀ and standard error values were obtained by iterative curve fitting using GraphPad Prism 2.0.

Radiolabelled Ligand Competition Receptor Binding Assays

Competition binding assays were performed using 50 pM ¹²⁵I-C5a or ¹²⁵I-C3a (NEN) on adherent C5aR-transfected or C5L2-transfected RBL cells in 96-well microtiter plates (55 000/well) at 4° C. as described previously (22). Competition curves were generated by pre-incubating adherent cells with increasing concentrations of unlabelled complement fragments. The IC₅₀, standard error values and linear regression analyses were obtained by using GraphPad Prism 2.0.

Fluorescent Ligand Binding/Uptake Assays

Cells were incubated with the indicated concentrations of FLUOS-labelled ASP or C3a for 30 minutes at 37° C. in binding buffer (23) and washed three times with cold binding buffer. Cells were then detached with 0.25% trypsin/0.02% EDTA in PBS, fixed with 1% paraformaldehyde, washed with PBS, and assayed by fluorescence-activated cell scanning.

Human Adipose Tissue and Murine 3T3-L1 Preadipocyte RT-PCR

Total RNA was isolated by Trizol extraction from freshly isolated samples of human adipose tissue or cultured 3T3-L1 cells. cDNA was produced from 3 μg of RNA by reverse transcriptase, and 4% of the reaction was amplified by PCR with 1.5 nM MgCl₂ and 0.01 mM tetramethyl ammonium chloride, under the following protocol: 1 min at 94° C., 1 min at 60° C., 2 min at 72° C. for 35 cycles. Primers for human C5L2 were: sense 5′-CCTGGTGGTCTACGGITCAG-3′ and antisense 5′-GGGCAGGATTTGTGTCTGTT-3′. Primers for murine C5L2 (Ensembl gene ID: ENSMUSG00000041388) were: sense 5′-ATGGCCGACTTGCTTTGT-3′ and antisense 5′-CCTTGGTCACCGCACTTTC-3′. Reaction products were separated on a 7.5% polyacrylamide gel and detected by silver staining (BioRad), and a 100 bp ladder (NEB) was used as standard. For sequencing, the PCR product was purified from a 1.2% agarose gel.

### EXAMPLE 1

We have previously shown that C5L2 has binding sites for C5a, C5a des-Arg⁷⁴, C4a and C3a (17). Here we show that the des-Arg⁷⁷ forms of C4a and C3a are also ligands for this receptor (FIGS. 1A, B, Table I) and can compete strongly with ¹²⁵I-C3a for C5L2 binding (FIG. 1A). In contrast, C4a des-Arg⁷⁷ and C3a des-Arg⁷⁷ cannot compete effectively with ¹²⁵I-C5a binding to C5L2 or CD88 (FIG. 1B, Table I). Although C3aR and C5L2 bind C3a with similar affinities, C3aR has no detectable affinity for C3a des-Arg⁷⁷ (Table I). Similarly, although C4a is a ligand at both C3aR and C5L2, with similar affinity for both receptors, C4a des-Arg⁷⁷ has a 1000-fold higher affinity for C5L2 than for C3aR (Table I). The data suggest that C5L2 may have two separate binding sites. One is a low affinity site, which preferentially binds ¹²⁵I-C3a, at which all of the complement fragments except C5a des-Arg⁷⁴ can compete with similar affinities. The second, high affinity, site preferentially binds ¹²⁵I-C5a, at which only C5a des-Arg⁷⁴ and, to a lesser extent, C4a, can compete.

Recombinant C3a des-Arg⁷⁷ can clearly compete with ¹²⁵I-C3a for binding to C5L2, so we then directly measured the affinity of C3a des-Arg⁷⁷ for C5L2, using protein purified from human plasma as ASP. ASP and purified human C3a were labelled with FLUOS. Increasing concentrations of ASP were incubated with HEK 293 cells transiently transfected with C5L2 and binding and uptake assessed by flow cytometry (FIG. 2A). FLUOS-ASP clearly binds to C5L2, with half maximal fluorescence intensity at approximately 3 nM, whereas mock-tansfected cells show no binding of ASP even at a high concentration of 10 nM. For comparison purposes, the binding of FLUOS-C3a and FLUOS-ASP to HEK 293 cells transiently transfected with C3aR is shown (FIG. 2B). Half maximal binding of FLUOS-C3a=2.5 nM, while ASP binding was not significantly different from basal (basal binding=100%; binding FLUOS-ASP=103%±14%, mean±SD, n=3). Similar data were obtained with cells stably-transfected with C3aR using FLUOS- and/or ¹²⁵I-labelled ligands (not shown). ¹²⁵I-ASP does not bind to C3aR-transfected cells and does not compete with ¹²⁵I-C3a (data not shown), as found previously (24). Thus, C5L2 has binding characteristics that overlap with both CD88 and C3aR but also the unique ability to bind ASP/C3a des-Arg⁷⁷.

### EXAMPLE 2

C3a des-Arg⁷⁷ binding enhances IgE-receptor-mediated degranulation in C5L2-transfected RBL cells. We have previously shown that C5a, C5a des-Arg⁷⁴, C4a and C3a binding to C5L2 does not stimulate an increase in intracellular [Ca²⁺] nor the degranulation of transfected RBL cells, possibly due to weak coupling to endogenous Gᵢ-like G proteins (17). However, addition of anaphylatoxic ligands does significantly enhance the secretory response to cross-linkage of the high affinity IgE receptor with IgE and antigen (17). As neither recombinant nor purified C3a des-Arg⁷⁷ (or any other ligand) is able to directly stimulate degranulation in C5L2-transfected RBL cells (data not shown), we have used the degranulation-enhancement assay to derive the dose-response relationship for C3a des-Arg⁷⁷ in RBL cells. Intact complement fragments can enhance the IgE-mediated degranulation response (FIG. 3A and (17)), with rank order of EC₅₀ values (C5a<C3a<C4a), in agreement with their affinities for the C3a binding site on C5L2 (Table I). In contrast, rank order of activation of C5L2 by the des-Arg ligands (EC₅₀ values C5a des-Arg⁷⁴<C3a des-Arg⁷⁷<<C4a des-Arg⁷⁷) (FIG. 3A) does not correlate with rank order of binding at this site (Table I). Human C3a des-Arg⁷⁷, purified from serum as acylation-stimulating protein, has an identical EC₅₀ to the recombinant C3a des-Arg⁷⁷, but appears to be somewhat less potent (FIG. 3A). The increased potency of the recombinant form may be a non-specific effect because it alone causes a small enhancement of degranulation in RBL cells transfected with an irrelevant receptor (FPRL-1) (FIG. 3B). C5a des-Arg⁷⁴, which cannot compete at all with ¹²⁵I-C3a for binding to C5L2 (FIG. 1B), has a lower EC₅₀ value than C5a (17) but is only a partial agonist (FIG. 3A). This suggests that the high affinity site couples less efficiently to the enhancement-signalling pathway than the low affinity site. However, C4a des-Arg⁷⁷, with a higher affinity for C5L2 than C3a des-Arg⁷⁷, is 230-fold less active (FIG. 3A). Thus, although binding to either site on C5L2 appears to activate the receptor, ligand binding per se does not necessarily cause activation. By contrast, neither C3a des-AXg⁷⁷ nor C4a des-Arg⁷⁷ can stimulate degranulation from RBL cells transfected with either CD88 or C3aR (data not shown).

### EXAMPLE 3

Both CD88 and C5L2 can couple through G_{αi} and G_{α16}. The C5a receptor CD88 can couple effectively to the pertussis toxin (PT)-sensitive G proteins Gᵢ₂ and Gᵢ₃ (25) and also to toxin-insensitive G_{q}-family member, G₁₆ (26,27). We reasoned that the weak stimulus-secretion coupling by C5L2 could be due to the absence of G₁₆ from RBL cells, which we tested by co-transfecting cells with human G_{α16} and either CD88 or C5L2. The bicistronic vector pIRES was used, to increase the likelihood that equal amounts of receptor and G protein would be expressed in transfected cells. With transfection of CD88 alone, increasing concentrations of PT inhibited the degranulation response (FIG. 4A). In co-transfected cells, CD88 clearly couples strongly to G_{α16}, and the degranulation response to C5a is resistant to doses of PT that could substantially inhibit degranulation in cells transfected with CD88 alone (FIG. 4B). At a higher dose of PT (10 ng/ml), a small inhibition of degranulation is observed, presumably due to stabilization of interactions between free subunits and ADP-ribosylated Gᵢ.

The enhancement of degranulation by purified human ASP/C3a des-Arg⁷⁷ in cells transfected with C5L2 alone can be inhibited by pre-treating cells with pertussis toxin (FIG. 4C), as found previously for the intact anaphylatoxins (17), suggesting the involvement of Gᵢ-like G proteins. In G₁₆+C5L2 co-transfected cells, treatment with high concentrations (1 M of intact or des-Arg complement fragments also does not directly stimulate any significant levels of degranulation (data not shown). However, enhancement of the degranulation response to IgE receptor cross-linking is seen in these cells after treatment with 10 ng/ml PT (FIG. 4D), although the pattern of activation is different to that observed in cells lacking G_{α16}. C4a appears to be inactive here, suggesting that coupling to different G proteins can subtly alter ligand-binding properties of C5L2. Thus, as with CD88, C5L2 appears to be able to couple with both the pertussis toxin sensitive G_{αi} and the pertussis toxin-insensitive G_{α16}.

### EXAMPLE 4

C5L2 mRNA is expressed in human adipose tissue and murine 3T3-L1 preadipocytes. Although C3a des-Arg⁷⁷ is regarded as biologically inactive in most myeloid systems, the acylation stimulating properties of this complement fragment are well documented (28). We therefore investigated the expression of C5L2 in human and murine adipose tissue, since both adipocytes and preadipocytes are known to respond to ASP/C3a des-Arg⁷⁷ by an increase in triglyceride synthesis (16). We used RT-PCR to detect expression using species-specific sets of primers to human and mouse adipose tissue mRNA. Both primer sets produced a band as seen on polyacrylamide electrophoresis gels at the size expected for a C5L2 transcript (FIG. 5). The human adipose tissue PCR product was extracted from an agarose gel and sequenced to confirm the authenticity of the transcript as that of C5L2. C3aR is not expressed in human adipose tissue (unpublished observations). By contrast RT-PCR of RNA from the human monocytic cell line U937 and non-transfected BEK 293 cells did not result in any PCR product using C5L2 primers (data not shown). On the other hand, U937 cells have been shown previously to express C3aR (23). C3aR has been shown in this report and elsewhere (8) to have no detectable affinity for C3a des-Arg⁷⁷. In contrast, C5L2 binds both ligands with high affinity and is expressed in adipose tissue, suggesting that it may be a functional C3a des-Arg⁷⁷/ASP receptor when expressed in the appropriate cell type.

### EXAMPLE 5

Stable transfection of the receptor reconstitutes activity in a non-responsive cell.

Using a plasmid that expresses C5L2, we have stably transfected this into HEK cells, cells which do not normally express C5L2. Using an antibody designed against the N terminal component of C5L2, we can demonstrate that this receptor is highly expressed in stably transfected HEK cells by FACSCAN (FIG. 14).

As shown in FIG. 2, HEK-C5L2 cells are also responsive to stimulation by ASP, increasing triglyceride synthesis in a concentration dependent manner. On the other hand, HEK non-transfected cells are not responsive to ASP, and neither HEK nor HEK-C5L2 are responsive to insulin (FIG. 15).

### EXAMPLE 6

Reconstituted activity can again be blocked using a signal transduction inhibitor that also blocks ASP activity in an endogenously expressing cell (3T3).

Previous work has demonstrated that that the ASP pathway involves intracellular signaling mediated via the protein kinase C pathway. Recently, we have demonstrated that PI-3 kinase is also involved in mediating ASP action, and addition of wortmanin, a PI-3 kinase inhibitor, not only blocks ASP action in normally responsive cells, but blocks ASP response in HEK-C5L2 stable transfectants (also shown in FIG. 15).

### EXAMPLE 7

Down regulation of endogenous expression using antisense oligonucleotides or interfering RNA (RNAi) also decreases C5L2 expression and response to ASP in normally responsive cells.

In order to evaluate C5L2 expression in endogenously expressing cells, we have used the antibody to evaluate endogenous expression, using non-immune serum to determine background. As shown in FIG. 14, auto-fluoresence in HSF cells is substantially greater than HEK cells (HEK-C5L2 are comparable to HEK. Nonetheless, expression of C5L2 can still be detected above the basal non-immune serum (FIG. 14).

Treatment of cells with a final concentration of 1, 2 or 4 μg/ml of various RNAi's, can reduce the expression of C5L2 to background levels (FIG. 16). A semi-quantitative assessment of receptor expression (based on % fluorescence intensity above non immune cells), demonstrates that varying conditions can gradually decrease the level of C5L2 immunoreactivity (FIG. 16).

Using various antisense oligonucleotides targeted to different regions of the mRNA at a final concentration of 0.2 or 0.24 μM, we can also reduce the response to ASP as assessed by triglyceride synthesis. As shown in FIG. 17, anti-sense to human C5L2 in HSF and to mouse C5L2 in 3T3, result in decreased response to ASP.

### REFERENCES

1. Kohl, J. (2001) Mol Immunol 38, 175-187
2. Bhatia, M., Brady, M., Shokuhi, S., Christmas, S., Neoptolemos, J. P., and Slavin, J. (2000) J Pathol 190, 117-125
3. Mukherjee, P., and Pasinetti, G. M. (2001) J Neurochem 77, 43-49
4. O'Barr, S. A., Caguioa, J., Gruol, D., Perkins, G., Ember, J. A., Hugli, T., and Cooper, N. R. (2001) J Immunol 166, 4154-4162
5. Mastellos, D., Papadimitriou, J. C., Franchini, S., Tsonis, P. A., and Lambris, J. D. (2001) J Imunol 166, 2479-2486
6. Schupf, N., Williams, C. A., Hugli, T. E., and Cox, J. (1983) J Neuroimmunol 5, 305-316
7. Schupf, N., Williams, C. A., Berlanan, A., Cattell, W. S., and Kerper, L. (1989) Brain Behav Immun 3, 28-38
8. Zwimer, J., Werfel, T., Wilken, H. C., Theile, E., and Gotze, O. (1998) Eur J Immunol 28, 1570-1577
9. Daffern, P. J., Pfeifer, P. H., Ember, J. A., and Hugli, T. E. (1995) J Exp Med 181, 2119-2127
10. Puschel, G. P., Nolte, A., Schieferdecker, H. L., Rothermel, E., Gotze, O., and Jungermann, K. (1996) Hepatology 24, 685-690
11. Klos, A., Bank, S., Gietz, C., Bautsch, W., Kohl, J., Burg, M., and Kretzschmar, T. (1992) Biochemistry 31, 11274-11282
12. Charriaut, C., Senik, A., Kolb, J. P., Barel, M., and Frade, R (1982) Proc Natl Acad Sci USA 79, 6003-6007
13. Fischer, W. H., Jagels, M. A, and Hugli, T. E. (1999) J Immunol 162, 453-459
14. Fischer, W. H., and Hugli, T. E. (1997) J Immunol 159, 4279-4286
15. Mousli, M., Hugli, T. E., Landry, Y., and Bronner, C. (1992) J Immunol 148, 2456-2461
16. Murray, I., Parker, R. A., Kirchgessner, T. G., Tran, J., Zhang, Z. J., Westerlund, J., and Cianflone, K. (1997) J Lipid Res 38, 2492-2501
17. Cain, S. A., and Monk, P. N. (2002) J Biol Chem 277, 7165-7169
18. Monk, P. N., Barker, M. D., Partridge, L. J., and Pease, J. E. (1995) J Biol Chem 270, 16625-16629
19. Paczkowski, N. J., Finch, A. M., Whitmore, J. B., Short, A. J., Wong, A. K., Monk, P. N., Cain, S. A., Fairlie, D. P., and Taylor, S. M. (1999) Br J Pharmacol 128, 1461-1466
20. Cain, S. A., Coughlan, T., and Monk, P. N. (2001) Biochemistry 40, 14047-14052
21. Monk, P. N., Bingham, B. R., Ahmad, T. B., and Helm, B. A. (1993) Receptor 3, 77-86
22. Monk, P. N., Barker, M. D., and Partridge, L. J. (1994) Biochim Biophys Acta 1221, 323-329
23. Crass, T., Raffetseder, U., Martin, U., Grove, M., Klos, A., Kohl, J., and Bautsch, W. (1996) Eur J Immunol 26, 1944-1950
24. Wilken, H. C., Rogge, S., Gotze, O., Werfel, T., and Zwirner, J. (1999) J Immunol Methods 226, 139-145
25. Sheth, B., Banks, P., Burton, D. R., and Monk, P. N. (1991) Biochem J 275 (Pt 3), 809-811
26. Shum, J. K., Allen, R. A., and Wong, Y. H. (1995) Biochem Biophys Res Commun 208, 223-229
27. Buhl, A. M., Eisfelder, B. J., Worthen, G. S., Johnson, G. L., and Russell, M. (1993) FEBS Lett 323, 132-134

28. Cianflone, K., Maslowska, M., and Sniderman, A. D. (1999) Semin Cell Dev Biol 10, 31-41

TABLE I
Comparison of competition binding data for human chemoattractant receptors expressed in RBL cells

| | Receptor: | | | | | | | | | | | |
|---|---|---|---|---|---|---|---|---|---|---|---|---|
| | CD88 | | | C3aR | | | C5L2 (¹²⁶I-C5a) | | | C5L2 (¹²⁵I-C3a) | | |
| | Ligand: | | | | | | | | | | | |
| | pD₂¹ ± SE | IC₅₀² | n³ | pD2 ± SE | IC₅₀ | n | pD2 ± SE | IC₅₀ | n | pD2 ± SE | IC₅₀ | n |
| C5a | 7.69 ± 0.03 | 20.5 | 15 | 5.54 ± 0.07 | 2900 | 3 | 8.09 ± 0.03 | 8.17 | 10 | 6.53 ± 0.16 | 293 | 3 |
| C5a des-Arg⁷⁴ | 6.39 ± 0.09 | 411 | 6 | 5.02 ± 0.18 | 9670 | 3 | 7.43 ± 10.07 | 36.9 | 3 | <3 | | 3 |
| C4a | 5.35 ± 0.09 | 4440 | 4 | 6.60 ± 0.08 | 250 | 3 | 5.42 ± 0.09 | 3790 | 4 | 6.31 ± 0.12 | 485 | 4 |
| C4a des-Arg⁷⁷ | ND⁴ | | | 4.99 ± 0.27 | 1.0 × 10⁵ | 2 | 4.72 ± 0.14 | 1.7 × 10⁵ | 2 | 6.75 ± 0.09 | 177 | 2 |
| C3a | 4.64 ± 0.33 | 2.3 × 10⁵ | 3 | 6.81 ± 0.05 | 155 | 12 | 4.64 ± 0.17 | 2.3 × 10⁵ | 3 | 6.78 ± 0.10 | 167 | 8 |
| C3a des-Arg⁷⁷ | ND | | | <4 | | 3 | 4.73 ± 0.16 | 1.8 × 10⁵ | 3 | 6.24 ± 0.10 | 526 | 3 |

¹pD₂ = −log IC50;
²IC₅₀ = concentration of unlabelled ligand resulting in 50% of maximal radioligand binding;
³n = number of separate experiments performed in triplicate;
⁴ND = assay not done.

1. A screening method for the identification of agents which modulate the interaction of the receptor C5L2 with C3-desArg⁷⁷/ASP comprising:
i) forming a preparation comprising a first and second polypeptide, or active fragments thereof, encoded by nucleic acid molecules selected from the group consisting of;
a) a first polypeptide encoded by a nucleic acid molecule comprising a nucleic acid sequence as represented in SEQ ID NO: 12;
b) a second polypeptide encoded by a nucleic acid molecule comprising a nucleic acid sequence as represented in SEQ ID NO: 16;
c) a polypeptide comprising a nucleic acid molecule which hybridises to the nucleic acid sequence as represented in SEQ ID NO: 12 and has the activity associated with the receptor C5L2;
d) a polypeptide comprising a nucleic acid molecule which hybridises to the nucleic acid sequence as represented in SEQ ID NO: 16 and has the activity associated with C3-desArg⁷⁷/ASP;
e) a polypeptide comprising a nucleic acid molecule which is degenerate because of the genetic code to the sequences in (a), (b), (c) or (d); and a candidate agent to be tested; and,
ii) detecting or measuring the effect of the agent on the interaction of the receptor C5L2 with C3-desArg⁷⁷/ASP.

2. The method of claim 1, wherein said nucleic acid molecule anneals under stringent hybridisation conditions to the sequence described in (a), (b), (c) or (d) above.

3. The method of claim 1, wherein said first polypeptide is encoded by a nucleic acid consisting of the sequence represented in SEQ ID NO: 12.

4. The method of claim 1, wherein said second polypeptide is encoded by a nucleic acid consisting of the sequence as represented in SEQ ID NO: 16.

5. The method of claim 1, wherein at least one polypeptide is modified by deletion, substitution or addition of at least one amino acid residue of the sequence represented in SEQ ID NO: 11 or 15.

6. The method of claim 1, wherein said polypeptides are expressed by a cell.

7. The method of claim 6, wherein said is cell is transfected/transformed with the receptor C5L2.

8. The method of claim 6, wherein said cell is a mammalian cell.

9. The method of claim 8, wherein said mammalian cell is a CHO, COS, HEK, 3T3, RBL; adipocyte or pre-adipocyte cell.

10. The method of claim 8, wherein said cell is an adipocyte or pre-adipocyte.

11. The method of claim 1, wherein said agent is a polypeptide, a peptide, an aptamer, an interfering RNA (RNAi) or an antisense oligonucleotide.

12. The method of claim 11, wherein said polypeptide is an antibody.

13. The method of claim 12, wherein said antibody is a polyclonal or monoclonal antibody.

14. The method of claim 12, wherein said antibody is specific for C5L2.

15. The method of claim 12, wherein said antibody is specific for C3a-desArg⁷⁷/ASP.

16. The method of claim 12, wherein said antibody is an agonists.

17. The method of claim 12, wherein said antibody is an antagonists.

18. The method of claim 11, wherein said peptide is an oligopeptide.

19. The method of claim 18, wherein said oligopeptide is at least 10, 20, 30, 40 or 50 amino acids in length.

20. The method of claim 18, wherein said peptide is modified.

21. The A method of claim 20, wherein said modification is acetylation and/or amidation and/or cyclisation.

22. The method of claim 11, wherein said aptamer comprises at least one modified nucleotide base.

23. The method of according to claim 11, wherein said RNAi molecule is derived from the nucleic acid molecule comprising a nucleic acid sequence selected from the group consisting of;
a) a nucleic acid sequence as represented by the sequence in SEQ ID NO: 12, or fragment thereof;
b) a nucleic acid sequence which hybridises to the nucleic acid sequences of SEQ ID NO: 12 and encodes a gene for the C5L2 receptor;
c) a nucleic acid sequence which comprise sequences which are degenerate as a result of the genetic code to the nucleic acid sequences defined in (a) and (b).

24. The method of claim 23, wherein of said RNAi molecule is between 10 nucleotide bases (nb)-1000 nb in length.

25. The method of claim 24, wherein said RNA molecule is 10 nb; 20 nb; 30 nb; 40 nb; 50 nb; 60 nb; 70 nb; 80 nb; 90 nb; or 100 bp in length.

26. The method of claim 24, wherein said RNA is 21 nb in length.

27. The method of claim 2 wherein said RNAi molecule comprises modified nucleotide bases.

28. The method of claim 11, wherein said antisense oligonucleotide is capable of hybridising to the nucleic acid molecule encoding the C5L2 receptor.

29. An agent obtained by the method of claim 1.

30. The agent of claim 29, wherein said agent is an agonist of C5L2 receptor activation by C3-desArg⁷⁷/ASP.

31. The agent of claim 29, wherein said agent is an antagonist of C5L2 receptor activation by C3-desArg⁷⁷/ASP.

32. The agent of claim 29, wherein said agent is a pharmaceutical.

33. The agent of claim 29, wherein said agent modulates fat deposition in adipocytes by modulating the interaction of the C5L2 receptor with C3-desArg⁷⁷/ASP.

34. (canceled)

35. A method of treating obesity in an animal comprising:
i) providing the agent of claim 29; and
ii) administering said agent to an animal.

36. The method of claim 35, wherein said animal is a human.

37. (canceled)

38. (canceled)

39. A method of treating obesity, comprising administration of a PI-3 inhibitor.

40. The method of claim 39, wherein the PI-3 inhibitor comprises wortmanin or a functional variant thereof.
